(19) United States
(12) Patent Application Publication
(10) Pub. No.: US 2019/0083422 A1
(43) Pub. Date: Mar. 21, 2019

(54) SENSE-33 COMPOSITIONS AND METHODS THEREOF FOR ENHANCING THE EXPRESSION AND/OR ACTIVITY OF NICOTINIC ACETYLCHOLINE RECEPTORS

(71) Applicant: Phaethon Psychis, Athens (GR)

(72) Inventor: Phaethon Psychis, Athens (GR)

(21) Appl. No.: 16/129,455

(22) Filed: Sep. 12, 2018

Related U.S. Application Data
(60) Provisional application No. 62/560,115, filed on Sep. 18, 2017.
(60) Provisional application No. 62/596,752, filed on Dec. 8, 2017.

(51) Int. Cl. A61K 31/137; A61P 25/28

(52) U.S. Cl. CPC A61K 31/137; A61P 25/28

(57) ABSTRACT

The present disclosure relates to compositions and methods thereof for enhancing the expression and/or function of Nicotinic Acetylcholine Receptors. Specifically, the compositions and methods of the present disclosure employ a family of structurally distinct Phenylalkylamine and diphenylalkylamine derivatives which are selective enhancers of the expression of nicotinic acetylcholine receptors, and/or specific nicotinic acetylcholine receptors subunits.

This application claims the benefit of U.S. provisional applications 62/560,115 and 62/596,752 filed Sep. 18, 2017 and Dec. 8, 2017, respectively.

### TECHNICAL FIELD

The present disclosure relates to compositions and methods thereof for enhancing the expression and/or function of Nicotinic Acetylcholine Receptors.

### BACKGROUND

Reduced function and/or expression of neuronal nicotinic Acetylcholine receptors (nAChRs), a subtype of Acetylcholine receptors (nAChRs) is related with the pathophysiology of healthy aging, but has also been associated with neurodegenerative disorders, including AD and schizophrenia. A natural decline in the expression of nicotinic receptors in the CNS is related to Ageing, accordingly, cognitive decline associated with healthy ageing, or dementia, may be correlated to the loss of nAChRs. Furthermore, the functionality of availability of nAChRs is reduced in the course of normal ageing. The high-affinity beta2 subunit of nAChR is inversely correlated with age, with age-related decline ranging up to 5% per decade over the adult lifespan. The importance of retaining the high-affinity nicotine binding sites of nAChRs to brain integrity has been demonstrated, where mice that lacked the beta2 subunit of the nAChRs experienced early onset neurodegeneration, and exhibited marked alteration in exploration, navigation and in organization of social behaviors. With the neurological and psychiatric disorders related to the function and/or expression of neuronal nicotinic Acetylcholine receptors fast becoming a social and financial burden, there is a need alleviate, or prevent, these conditions. The present disclosure provides pharmaceutical compositions and methods thereof for the treatment and/or prophylaxis of neuronal nicotinic Acetylcholine receptors related disorders.

### SUMMARY

The pharmaceutical compositions and methods thereof described herein provide treatment and/or prophylaxis of neuronal nicotinic Acetylcholine receptors related disorders. In some embodiments, the present disclosure provides a pharmaceutical composition comprising a therapeutically effective amount of at least one compound of general formula (I). In some embodiments, the compound of general formula (I) is selected from: a deuterated analog of 2-[(1R)-1-phenyl-3-[(1-phenylethyl)amino]propyl]phenol, 2-[(1S)-1-phenyl-3-[(1-phenylethyl)amino]propyl]phenol, and 3,3-diphenyl-N-(1-phenylethyl)propan-1-amine. In some embodiments, the compound of general formula (I) is 3,3-diphenyl-N-(1-phenylethyl)propan-1-amine.

In some embodiments, the present disclosure provides a method of treatment of a subject in need, wherein said subject is suffering from a disease related to a dysfunction of the neuronal nicotinic acetylcholine receptor system, wherein the method comprises administering to said subject a therapeutically effective amount of a pharmaceutical composition selected from the pharmaceutical compositions described above. In some embodiments, said diseases is a is a reduction in the density of neuronal nicotinic acetylcholine receptors in the brain. In some embodiments, said diseases is a is age related cognitive decline. In some embodiments, said subject is in need of cognitive enhancement during reinforced learning, working tasks, and decision-making processes.

In some embodiments, the present disclosure provides a method of providing prophylaxis to a subject, wherein said subject is likely to suffer from a disease related to a dysfunction of the neuronal nicotinic acetylcholine receptor system, and wherein the method comprises administering to said subject a therapeutically effective amount of a pharmaceutical composition selected from the pharmaceutical compositions described above. In some embodiments, said diseases is a is a reduction in the density of neuronal nicotinic acetylcholine receptors in the brain. In some embodiments, said diseases is a is age related cognitive decline. In some embodiments, said subject is in need of cognitive enhancement during reinforced learning, working tasks, and decision-making processes.

In any of the methods of the disclosure, the composition may be administered to the subject by any method known in the art (e.g., those described herein). In certain embodiments, the composition is administered by a route selected from the group consisting of oral, rectal, parenteral (e.g., intravenously or intramuscularly), cutaneous, topical, nasal, vaginal, inhalant, skin (patch), and ocular.

By “treating” is meant ameliorating at least one symptom of a condition or disease in a subject having the condition or disease (e.g., a subject diagnosed with a neurological disorder), as compared with an equivalent untreated control. Such reduction in the symptom (e.g., a reduction in By “neurological disorder” is meant any disease, condition and disorder of the central nervous system. Neurological disorders according to the present disclosure are, but not limited to, Alzheimer's disease, Parkinson's Disease, amyotrophic lateral sclerosis, epilepsy, autism, rett syndrome, Huntington's Disease, stroke, spinal cord injury, traumatic brain injury, Lewy body dementia, multiple sclerosis, Pick's disease, Niewmann-Pick disease, Creutzfeld-Jakob disease, Guillain-Barre syndrome, Bell's palsy, diabetic neuropathy, amyotrophic lateral sclerosis, amyloid angiopathy, cerebral amyloid angiopathy, systemic amyloidosis, hereditary cerebral hemorrhage with amyloidosis of the Dutch type, inclusion body myositis, mild cognitive impairment, complications due to stroke, head trauma, or spinal injury, or other injuries to the brain, peripheral nervous, central nervous, or neuromuscular system, acute spinal cord and brain injury, and demyelinating diseases, such as multiple sclerosis.

By “effective amount” is meant an amount of a compound required to treat, treat prophylactically, or reduce disease or disorder in a clinically relevant manner. For example, a effective amount of an active compound used to according to the present disclosure varies depending upon the manner of administration, the age, body weight, and general health of the subject. Ultimately, the prescribers will decide the appropriate amount and dosage regimen.

By “subject” is meant a human or non-human animal (e.g., a mammal).

By “increase” is meant an amount greater by at least 5%, I 0%, 25%, 50%, I 00%, 150%, 200%, 500%, or I 000%.

### BRIEF DESCRIPTION OF THE DRAWING

FIG. 1 depicts Fendiline derivatives according to James Wilkinson et al., (reference 3).

### DETAILED DESCRIPTION

Diseases or conditions related to reduction in function and/or/expression of neuronal nicotinic Acetylcholine receptors (nAChR) are various and include, but are not limited to, mild memory loss, Alzheimer's, and schizophrenia. Studies have recently revealed the importance of retaining the high-affinity nicotine binding sites to brain integrity. Mice that lacked the beta2 subunit of nAChRs experienced early onset neurodegeneration, and exhibited marked alteration in exploration and navigation and organization of social behaviors. Neuronal nicotinic Acetylcholine receptors are receptor proteins that respond to the neurotransmitter acetylcholine. Nicotinic receptors also respond to drugs, including the nicotinic receptor agonist nicotine. They are found in the central and peripheral nervous system, muscle, and many other tissues of many organisms, including humans. The nicotinic receptors are considered cholinergic receptors, since they respond to acetylcholine.

Endogenous Ligands and Drug Interactions

There are at least six human neuronal nAChR subunits: a3,4,5,7 and B2,3,4. nAChRs present in different body locations may be composed of distinct compositions of subunits. In neuronal nAChRs, the binding site is located at the interface of an a and a B subunit or between two a subunits in the case of a7 receptors. nAChR respond to the neurotransmitter acetylcholine. Choline is also an endogenous agent of importance to nAChR function. In addition to its role as a metabolic precursor to the principal endogenous cholinergic neurotransmitter, acetylcholine, choline can also act to modify receptor activation and expression in distinct ways, and corresponds with an increase in B2 subunit protein expression. When co-applied with the pro-inflammatory cytokine tumor necrosis factor a (TNF Alpha), choline-mediated up-regulation of B2 subunit protein expression is strongly increased further through a mechanism that includes an increase in both a4 and B2 subunits expression.

Choline can also modify the function of certain other nAChR's subunits such a3, a4, or B4 (Gahring L C, Vasquez-Opazo G A, Rogers S W. Choline Promotes Nicotinic Receptor a4+B2 Up-regulation. The Journal of Biological Chemistry. 2010; 285(26):19793-19801. doi:10.1074/jbc.M110.108803).

Medications Related to Cholinergic System Dysfunction

In addition to Choline, or acetylcholine, nAChRs also responds to various drugs, including nicotinic drugs. There is a large body of evidence indicating that nicotinic drugs affect learning and memory. Nicotine and other nicotinic agonists can improve cognitive and psychomotor function, whereas nicotinic antagonists lead to cognitive impairment.

Acetylcholine is essential for a variety of complex behaviors such as the performance of attention and learning tasks, and cholinergic deficits are central to the etiology of dementias.

Treatments to improve cholinergic neurotransmission are the standard-of-care for diseases such as Alzheimer's (AD). However, the therapeutic benefits of the treatments available to date do not provide an effective solution for the treatment, or prevention, of dementia, AD, or other nAChRs related diseases. Further, as most available drugs act as Cholinesterase Inhibitors, they generate side-effects as a result of muscarinic overstimulation.

There are currently five medications which are FDA-approved for the treatment of Alzheimer's Disease. All five are cholinesterase inhibitors: tacrine (COGNEX®; Sciele), donepezil (ARICEPT®; Pfizer), rivastigmine (EXELON®; Novartis), galantamine (RAZADYNE®), and memantine (NAMENDA®; Forest Ortho-McNeil-Janssen). Donepezil, rivastigmine, and galantamine are successors to tacrine, a first-generation compound rarely prescribed because of the potential for hepatotoxicity. Donepezil (Aricept) and galantamine, while generating a certain increase in the nAChR density, fail to produce detectable behavioral and cognitive improvements. Both drugs act as allosteric potentiating ligands of the nAChRs. Sigma ligand receptor modulators have emerged as novel therapeutic treatment and some have reached Phase II clinical trials for CNS indications. For example, Cyr-101/MT-210 (Cyrenaic Pharmaceuticals; Mitsubishi) is a sigma-2 receptor antagonist in phase IIa clinical trials for the condition of schizophrenia. Sigma-1 receptor ligands are in clinical trials for treatments of various CNS indications. Anavex 2-73 is a sigma-1 receptor agonist that antagonistically interacts with muscarinic cholinergic receptors such as M1/2/3 (M1/2/3 are subtypes of muscarinic cholinergic receptors which usually are expressed at different body areas), and acts as an antagonist with respect to various ion channels (NMDAR, Na+, Ca++). However, recent phase II clinical trial involving ANAVEX 2-73 failed to provide statistically meaningful results in cognitive improvement. A co administration of ANAVEX 2-73 with Donepezil also failed to provide significant improvement in cognitive abilities.

Another clinical approach involves the use of monoclonal antibodies to fight probable disease contributors, such as beta-amyloid peptide accumulations. Currently available data is inconclusive to support the use of this approach (Kerchner et al, 2010, Bapineuzumab, Expert Opin Biol Ther., 10(7):1121-1130).

There is an urgent need to provide pharmaceutical compositions that have pharmacological activity towards the cholinergic system that is both effective and selective, while having good “drugability” properties, i.e. good pharmaceutical properties related to administration, distribution, metabolism and excretion, in order to address the unmet need of sufferers of age-related cognitive decline and dementia related cognitive impairment. It is an object of the present disclosure to provide such pharmaceutical compositions.

Hence, the goal of the compositions and methods thereof disclosed herein is to selectively enhance the expression and functionality of selective nAChRs subtypes, while stimulating the production of endogenous nAChRs agonists.

Cholinergic System Dysfunction

Recent study of healthy ageing population has revealed an age-related decline in B2-nAChR in both cortical and subcortical regions.

Age related cognitive decline is likely associated with reduced receptor nicotinic receptor availability. Age related cognitive decline occurs in healthy (normal) ageing population, and sometimes is not associated with evidence of significant cognitive impairment, as has been indicated by the use of the Folstein Mini-Mental State Examination (MMSE.

The phenotype of normal cognitive ageing is well described. Some mental capabilities are well maintained into old age. From early adulthood, there are declines in mental domains such as processing speed, reasoning, memory and executive functions, some of which is underpinned by a decline in a general cognitive factor. The determinants of the differences in age-related cognitive decline are not fully understood.

Cholinergic system dysfunction is also associated with numerous neurodegenerative diseases such as Alzheimer's, Parkinson's and Huntington, as well as psychiatric disorders such as schizophrenia. These conditions are characterized by cognitive, behavioral and motor disabilities strongly correlated to neuronal nicotinic cholinergic circuit dysfunction.

General Embodiments of the Present Disclosure

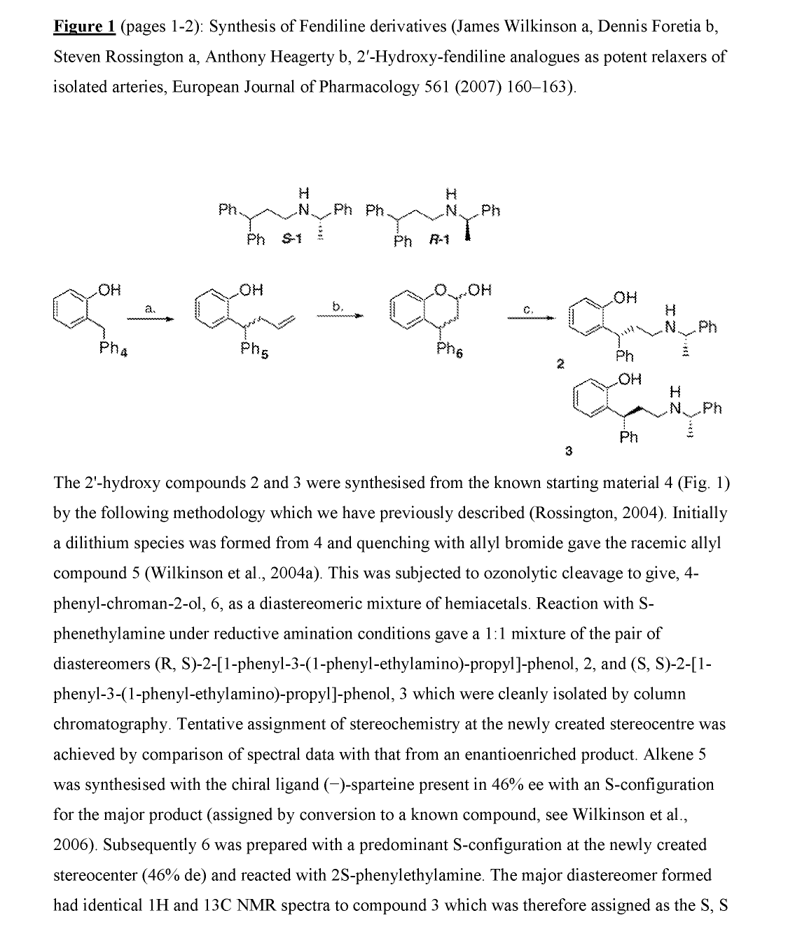

In some embodiments, the present disclosure provides a method of treatment, and/or prophylaxis, of an ageing subject suffering from cognitive decline, or an ageing subject likely to suffer from cognitive decline, the said method comprising administering to the patient in need a therapeutically effective amount of a composition comprising at least one compound selected from Table(1), or FIG. 1. In some embodiments, said cognitive decline is associated with decline in the density, or expression, of B2-nAChR in the brain.

In some embodiments, the present disclosure provides a method of treatment, and/or prophylaxis, of an ageing subject suffering from cognitive decline, or an ageing subject likely to suffer from cognitive decline, the said method comprising administering to the patient in need a therapeutically effective amount of a composition comprising at least one compounds according to formula (I). In the present disclosure, compounds according to formula (I) are described in Table(1). In some embodiments, said cognitive decline is associated with decline in the density, or expression, of B2-nAChR in the brain.

In some embodiments, the compositions disclosed herein act to provide treatments for cognitive and behavioral impairment in a wide spectrum of neurodegenerative diseases with diverse etiologies.

In some embodiments, the compositions and methods thereof disclosed herein provide treatment, or prophylaxis, of age-related cognitive decline, cognitive impairment and executive functioning.

Decision-making and executive functioning is an essential component of everyday life, and is commonly disabled in normal ageing as well as a myriad of psychiatric conditions, including bipolar and impulsive control disorders, addiction and pathological gambling, or schizophrenia.

Impaired decision-making is a symptomatic feature of a number of diseases with diverse etiology, or the normal process of ageing.

Generally, the present disclosure relates to compounds of general formula (I), described herein in Table(1). In some embodiments, the said compounds of general formula (I) enhance the expression of MicroRNA22.

MIR22HG is a long non-coding (lnc) RNA highly expressed in human cells in some diseases or stress conditions. Interestingly, lncRNA MIR22HG hosts, in the second exon, the micro-RNA22 (MIR22). Micro-RNAs (miRNAs), such as MIR22, are noncoding RNAs of 18 to 24 nucleotides that regulate the stability and translational efficiency of complementary target mRNAs. Chang et al., (2008) mapped the MIR22 gene to an exon of the MIR22HG (C17ORF91) gene. MicroRNA-22 is a circadian rhythm oscillating MicroRNA.

Studies have identified PTEN and SIRT1 as direct targets of MicroRNA22 (Targetscan). The inverse correlation between the miR-22 expression and the protein expression of SIRT1 reveals a MICRORNA22/SIRT1 loop. SIRT1 is a NAD(+)-dependent protein deacetylase that governs many physiological pathways, including circadian rhythm in peripheral tissues. Studies have shown that SIRT1 in the brain governs central circadian control by activating the transcription of the two major circadian regulators, BMAL1 and CLOCK. This activation comprises an amplifying circadian loop involving SIRT1, PGC-1a, and Nampt.

Derivatives of formula (I) according to the present disclosure are known in the art. In James Wilkinson et al., (James Wilkinson a, Dennis Foretia b, Steven Rossington a, Anthony Heagerty b, 2′-Hydroxy-fendiline analogues as potent relaxers of isolated arteries, European Journal of Pharmacology 561 (2007) 160-163) said derivatives are described as vasodilators, in WO2014031755A1 as having anticancer activity, and in US20180193289 as having pharmacological activity useful, inter alia, in the prophylaxis and/or treatment of neurodegenerative diseases, Neuropathic pain and Cancer. Fendiline, one of the derivatives of general formula (I), has been described as an L type Calcium Channel Blocker, a Sigma 1 Receptor agonist, a Sigma 1 Receptor antagonist, and as a Sigma 2 Receptor antagonist.

The present inventor describes herein the hitherto unknown and surprising positive effect of the said compounds of general formula (I) on the expression of genes that encodes the neuronal nicotinic acetylcholine receptor (nAChR), and on the MicroRNA22/SIRT1 pathway.

In some embodiments of the present disclosure, compounds of general formula (I) include Fendiline and its derivatives. Fendiline and its derivatives, according to the present disclosure, are described in US application US20150344407A1 hereby incorporated in its entirety by reference, in Table(1), FIG. 1, and herein. Fendiline and its derivatives, according to FIG. 1 were described in James Wilkinson, Dennis Foretia, Steven Rossington, Anthony Heagerty, John Leonard, Nigel Hussain, Clare Austin, 2′-Hydroxy-fendiline analogues as potent relaxers of isolated arteries, European Journal of Pharmacology, Volume 561, Issues 1-3, 2007, Pages 160-163, ISSN 0014-2999, https://doi.org/10.1016/j.ejphar.2007.01.039. (http://www.sciencedirect.com/science/article/pii/S0014299907000866) hereby incorporated in its entirety by reference.

The terms “derivatives of formula (I)”, “derivatives of general formula (I)”, “derivatives according to formula (I)”, “derivatives according to general formula (I)”, “compounds of formula (I)”, “compounds of general formula (I)”, “compounds according to formula (I)”, and “compounds according to general formula (I)” are used interchangeably herein.

The present inventor discovered that a family of structurally distinct Phenylalkylamine and diphenylalkylamine derivatives, according to general formula (I), are particularly selective enhancers and modulators of the nicotinic acetylcholine receptors. Specifically, it was found that Phenylalkylamine derivatives according to general formula (I) enhance the expression of CHRNB2.

The present inventor has also discovered that derivatives of general formula (I) enhance the expression of Choline Kinase, the first enzyme in the Kennedy pathway, metabolizing phosphocholine to free choline which is an endogenous agonist of Nicotinic Acetylcholine Receptors.

Therefore, in some embodiments the present disclosure relates to compositions and methods thereof used for the regulation the expression of CHRNB2 gene that encodes the Cholinergic Receptor Nicotinic Beta 2 Subunit.

In some embodiments, the present disclosure relates to compositions at least one compound according to general formula (I) that enhance the expression and functionality of neuronal nicotinic acetylcholine receptors (nAChRs).

In some embodiments, the present disclosure provides compositions comprising at least one compound according to general formula (I) for the treatment of disorders related to the dysfunction of the cholinergic system.

In some embodiments, the present disclosure provides compositions comprising at least one compound according to general formula (I) for the treatment of disorders related to reduction in the expression of neuronal nAChRs.

In some embodiments, the present disclosure provides compositions comprising at least one compound according to general formula (I) to enhance the expression of Nicotinic Acetylcholine Receptors.

In some embodiments, the present disclosure provides compositions comprising at least one compound according to general formula (I) for manufacturing a medicament for the treatment of diseases related to the cholinergic system dysfunction. when said compositions are used for prophylaxis and/or treatment of diseases related to the cholinergic system dysfunction.

In some embodiments, the present disclosure provides a method of treatment and/or prophylaxis of a subject suffering from a disease related to the cholinergic system dysfunction, or is likely to suffer from diseases related to the cholinergic system dysfunction, the said method comprising administering to said subject in need a composition comprising at least one compound according to general formula (I).

In preferred embodiment the present disclosure relates to the diseases and conditions associated with the reduction of b2-nAChRs subunit and more particularly in the age-related decline in B2-nAChR which is strongly correlated with the cognitive decline associated with normal aging.

In some aspects, the disclosure provides a method of selectively inhibiting expression of an aberrantly expressed gene comprising disrupting the function of an enhanceosome (Maniatis and Thanos 1995) associated with the aberrantly expressed gene. In some embodiments the said gene is an oncogene. In some embodiments the oncogene is selected from the group consisting of c-MYC and IRF4. In some embodiments, the said gene has a disease associated DNA sequence variation such as a SNP. In some embodiments, the said disease is Alzheimer's disease, and the gene is BIN1 (e.g., having a disease associated DNA sequence variation such as a SNP). In some embodiments, the said disease is type 1 diabetes, and the said gene is associated with a primary Th cell (e.g., having a disease associated DNA sequence variation such as a SNP). In some embodiments, the said disease is systemic lupus erythematosus, and the gene plays a key role in B cell biology (e.g., having a disease associated DNA sequence variation such as a SNP). In some embodiments, the said gene is associated with a hallmark characteristic of the cell. In some embodiments, the said gene is aberrantly expressed or is associated with a DNA variation such as a SNP. In some embodiments, the cell or tissue includes one of the following: mammalian cell, e.g., human cell; fetal cell; embryonic stem cell or embryonic stem cell-like cell, e.g., cell from the umbilical vein, e.g., endothelial cell from the umbilical vein; muscle, e.g., myotube, fetal muscle; blood cell, e.g., cancerous blood cell, fetal blood cell, monocyte; B cell, e.g., Pro-B cell; brain, e.g., astrocyte cell, angular gyms of the brain, anterior caudate of the brain, cingulate gyms of the brain, hippocampus of the brain, inferior temporal lobe of the brain, middle frontal lobe of the brain, brain cancer cell; T cell, e.g., naïve T cell, memory T cell; CD4 positive cell; CD25 positive cell; CD45RA positive cell; CD45RO positive cell; IL-17 positive cell; a cell that is stimulated with PMA; Th cell; Th17 cell; CD255 positive cell; CD127 positive cell; CD8 positive cell; CD34 positive cell; duodenum, e.g., smooth muscle tissue of the duodenum; skeletal muscle tissue; myoblast; stomach, e.g., smooth muscle tissue of the stomach, e.g., gastric cell; CD3 positive cell; CD14 positive cell; CD19 positive cell; CD20 positive cell; CD34 positive cell; CD56 positive cell; prostate, e.g., prostate cancer; colon, e.g., colorectal cancer cell; crypt cell, e.g., colon crypt cell; intestine, e.g., large intestine; e.g., fetal intestine; bone, e.g., osteoblast; pancreas, e.g., pancreatic cancer; adipose tissue; adrenal gland; bladder; esophagus; heart, e.g., left ventricle, right ventricle, left atrium, right atrium, aorta; lung, e.g., lung cancer cell; skin, e.g., fibroblast cell; ovary; psoas muscle; sigmoid colon; small intestine; spleen; thymus, e.g., fetal thymus; breast, e.g., breast cancer; cervix, e.g., cervical cancer; mammary epithelium; liver, e.g., liver cancer; DND41 cell; GM12878 cell; H1 cell; H2171 cell; HCC1954 cell; HCT-116 cell; HeLa cell; HepG2 cell; HMEC cell; HSMM tube cell; HUVEC cell; IMR90 cell; Jurkat cell; K562 cell; LNCaP cell; MCF-7 cell; MM1S cell; NHLF cell; NHDF-Ad cell; RPMI-8402 cell; U87 cell; VACO 9M cell; VACO 400 cell; or VACO 503 cell. In some embodiments, the said gene comprises a disease-associated variation related to rheumatoid arthritis, multiple sclerosis, systemic scleroderma, primary biliary cirrhosis, Crohn's disease, Graves disease, vitiligo and atrial fibrillation.

In some embodiments, the present disclosure relates to active substance compositions which comprise compounds according to Table(1):

Table (1): compounds of general formula (I) and Derivatives of general formula (I)

Some preferred variants of the Nicotinic Acetylcholine Receptors modulators, are listed in Table(1):
3,3-diphenyl-N-(1-phenylethyl)propan-1-amine (SENS-33), (R)-enantiomer of fendiline, (S)-enantiomer of fendiline,
2-[(1R)-1-phenyl-3-[(1-phenylethyl)amino]propyl]phenol, and
2-[(1S)-1-phenyl-3-[(1-phenylethyl)amino]propyl]phenol.

The pharmaceutical composition of the disclosure may be formulated for its simultaneous, separate or sequential administration.

In a preferred embodiment of the present disclosure, cholinergic system dysfunction refers specifically to conditions related to “Decision Making Impairment” and “Mild Cognitive Impairment” and “Neuropsychiatric symptoms”. The embodiments described above are additionally defined hereinafter and in the claims.

“Decision Making Impairment” refers to impairment in the process of decision-making (also spelled decision making) which is regarded as the cognitive process resulting in the selection of a belief or a course of action among several alternative possibilities. Every decision-making process produces a final choice, which may or may not prompt action.

“Mild Cognitive Impairment” (also referred to herein as mild cognitive impairment) also known as isolated memory impairment, is a neurological disorder that occurs in older adults which involves cognitive impairments with minimal impairment in instrumental activities of daily living. Mild cognitive impairment involves the onset and evolution of cognitive impairments beyond those expected based on the age and education of the individual, but which are not significant enough to interfere with their daily activities.

The term “Neuropsychiatric symptoms” refers to herein, without limitation, to psychosis (delusions and hallucinations), affective and behavioral changes (such as depressive mood), anxiety, irritability/lability, apathy, euphoria, disinhibition, agitation/aggression, aberrant motor activities, sleep disturbance, and eating disorder.

The term “reinforced learning” generally refers to the process by which decisions are made based on the evaluation of past experiences and the expectation of future outcomes that have been mentally created and maintained in working memory.

The term “working task” generally refers to any piece of work to be done or undertaken.

In the context of the present disclosure, the following terms have the meaning detailed below. “Alkyl” refers to a straight or branched hydrocarbon chain radical containing no unsaturation, and which is attached to the rest of the molecule by a single bond. Typical alkyl groups have from 1 to about 12, 1 to about 8, or 1 to about 6 carbon atoms, e. g., methyl, ethyl, n-propyl, i-propyl, n-butyl, t-butyl, n-pentyl, etc. Alkyl radicals may be optionally substituted by one or more substituents such as aryl, halo, hydroxy, alkoxy, carboxy, cyano, carbonyl, acyl, alkoxycarbonyl, heterocyclyl, amino, nitro, mercapto, alkylthio, etc. If substituted by aryl, it corresponds to an “arylalkyl” radical, such as benzyl or phenethyl. If substituted by heterocyclyl, it corresponds to a “heterocyclylalkyl” radical.

“Alkenyl” refers to a straight or branched hydrocarbon chain radical containing at least two carbon atoms and at least one unsaturation, and which is attached to the rest of the molecule by a single bond. Typical alkenyl radicals have from 2 to about 12, 2 to about 8 or 2 to about 6 carbon atoms. In a particular embodiment, the alkenyl group is vinyl, 1-methyl-ethenyl, 1-propenyl, 2-propenyl, or butenyl.

“Alkynyl” refers to a straight or branched hydrocarbon chain radical containing at least two carbon atoms and at least one carbon-carbon triple bond, and which is attached to the rest of the molecule by a single bond. Typical alkynyl radicals have from 2 to about 12, 2 to about 8 or 2 to about 6 carbon atoms. In a particular embodiment, the alkynyl group is ethynyl, propynyl (e.g. 1-propynyl, 2-propynyl), or butynyl (e.g. 1-butynyl, 2-butynyl, 3-butynyl).

“Cycloalkyl” refers to an alicyclic hydrocarbon. Typical cycloalkyl radicals contain from 1 to 3 separated and/or fused rings and from 3 to about 18 carbon atoms, preferably from 3 to 10 carbon atoms, such as cyclopropyl, cyclohexyl or adamantyl. In a particular embodiment, the cycloalkyl radical contains from 3 to about 6 carbon atoms.

“Aryl” refers to single and multiple ring radicals, including multiple ring radicals that contain separate and/or fused aryl groups. Typical aryl groups contain from 1 to 3 separated or fused rings and from 6 to about 18 carbon ring atoms, such as phenyl, naphthyl (e.g. 2-naphthyl), indenyl, fenanthryl or anthracyl radical. The aryl radical may be optionally substituted by one or more substituents such as hydroxy, mercapto, halo, alkyl, phenyl, alkoxy, haloalkyl, nitro, cyano, dialkylamino, aminoalkyl, acyl, alkoxycarbonyl, etc.

“Heterocyclyl” refers to a stable, typically 3- to 18-membered, ring radical which consists of carbon atoms and from one to five heteroatoms selected from the group consisting of nitrogen, oxygen, and sulfur, preferably a 4- to 15-membered ring with one or more heteroatoms, preferably a 4- to 8-membered ring with one or more heteroatoms, more preferably a 5- or 6-membered ring with one or more heteroatoms. It may be aromatic or not aromatic. For the purposes of this disclosure, the heterocycle may be a monocyclic, bicyclic or tricyclic ring system, which may include fused ring systems; and the nitrogen, carbon or sulfur atoms in the heterocyclyl radical may be optionally oxidized; the nitrogen atom may be optionally quaternized; and the heterocyclyl radical may be partially or fully saturated or aromatic. Examples of such heterocycles include, but are not limited to, azepines, benzimidazole, benzothiazole, furan, isothiazole, imidazole, indole, piperidine, piperazine, purine, quinoline, thiadiazole, tetrahydrofuran, coumarine, morpholine; pyrrole, pyrazole, oxazole, isoxazole, triazole, imidazole, etc.

“Alkoxy” refers to a radical of the formula —OR a where R a is an alkyl radical as defined above having one or more (e.g., 1, 2, 3 or 4) oxygen linkages and typically from 1 to about 12, 1 to about 8 or 1 to about 6 carbon atoms, e. g., methoxy, ethoxy, propoxy, etc.

“Aryloxy” refers to a radical of formula —O-aryl, where aryl is as previously defined. Some examples of aryloxy compounds are —O-phenyl (i.e. phenoxy), —O-p-tolyl, —O-m-tolyl, —O-o-tolyl or —O-naphthyl.

“Amino” refers to a radical of the formula —NH 2, —NHR a or —NR aR b, optionally quaternized. In an embodiment of the disclosure each of R a and R b is independently selected from hydrogen and an alkyl radical as defined above. Therefore, examples of amino groups are, methylamino, ethylamino, dimethylamino, diethylamino, propylamino, etc.

Halogen”, “halo” or “hal” refers to bromo, chloro, iodo or fluoro.

“Fused ring system” refers to a polycyclic ring system that contains fused rings. Typically, the fused ring system contains 2 or 3 rings and/or up to 18 ring atoms. As defined above, cycloalkyl radicals, aryl radicals and heterocyclyl radicals may form fused ring systems. Thus, fused ring system may be aromatic, partially aromatic or not aromatic and may contain heteroatoms. A spiro ring system is not a fused-polycyclic by this definition, but fused polycyclic ring systems of the disclosure may themselves have spiro rings attached thereto via a single ring atom of the system. Examples of fused ring systems are, but are not limited to, adamantyl, naphthyl (e.g. 2-naphthyl), indenyl, fenanthryl, anthracyl, pyrenyl, benzimidazole, benzothiazole, etc.

Unless otherwise stated specifically in the specification, all the groups may be optionally substituted, if applicable. References herein to substituted groups in the compounds of the present disclosure refer to the specified moiety that may be substituted at one or more (e.g., 1, 2, 3 or 4) available positions by one or more suitable groups, e. g., halogen such as fluoro, chloro, bromo and iodo; cyano; hydroxyl; nitro; azido; acyl, such as alkanoyl, e.g. a C 1-6 alkanoyl group, and the like; carboxamido; alkyl groups including those groups having 1 to about 12 carbon atoms or from 1 to about 6 carbon atoms and more preferably 1-3 carbon atoms; alkenyl and alkynyl groups including groups having one or more (e.g., 1, 2, 3 or 4) unsaturated linkages and from 2 to about 12 carbon or from 2 to about 6 carbon atoms; alkoxy groups having one or more (e.g., 1, 2, 3 or 4) oxygen linkages and from 1 to about 12 carbon atoms or 1 to about 6 carbon atoms; aryloxy such as phenoxy; alkylthio groups including those moieties having one or more (e.g., 1, 2, 3 or 4) thioether linkages and from 1 to about 12 carbon atoms or from 1 to about 6 carbon atoms; alkylsulfinyl groups including those moieties having one or more (e.g., 1, 2, 3 or 4) sulfinyl linkages and from 1 to about 12 carbon atoms or from 1 to about 6 carbon atoms; alkylsulfonyl groups including those moieties having one or more (e.g., 1, 2, 3 or 4) sulfonyl linkages and from 1 to about 12 carbon atoms or from 1 to about 6 carbon atoms; aminoalkyl groups such as groups having one or more (e.g., 1, 2, 3 or 4) N atoms and from 1 to about 12 carbon atoms or from 1 to about 6 carbon atoms; carbocylic aryl having 6 or more carbons, particularly phenyl or naphthyl and aralkyl such as benzyl. Unless otherwise indicated, an optionally substituted group may have a substituent at each substitutable position of the group, and each substitution is independent of the other.

The term “salt” must be understood as any form of a compound used in accordance with this disclosure in which said compound is in ionic form or is charged and coupled to a counter-ion (a cation or anion) or is in solution. This definition also includes quaternary ammonium salts and complexes of the molecule with other molecules and ions, particularly, complexes formed via ionic interactions. The definition includes in particular physiologically acceptable salts; this term must be understood as equivalent to “pharmacologically acceptable salts” or “pharmaceutically acceptable salts”.

The term “pharmaceutically acceptable salts” in the context of this disclosure means any salt that is tolerated physiologically (normally meaning that it is not toxic, particularly, as a result of the counter-ion) when used in an appropriate manner for a treatment, applied or used, particularly, in humans and/or mammals. These physiologically acceptable salts may be formed with cations or bases and, in the context of this disclosure, are understood to be salts formed by at least one compound used in accordance with the disclosure-normally an acid (deprotonated)-such as an anion and at least one physiologically tolerated cation, preferably inorganic, particularly when used on humans and/or mammals. Salts with alkali and alkali earth metals are preferred particularly, as well as those formed with ammonium cations (NH 4+). Preferred salts are those formed with (mono) or (di)sodium, (mono) or (di)potassium, magnesium or calcium. These physiologically acceptable salts may also be formed with anions or acids and, in the context of this disclosure, are understood as being salts formed by at least one compound used in accordance with the disclosure-normally protonated, for example in nitrogen-such as a cation and at least one physiologically tolerated anion, particularly when used on humans and/or mammals. This definition specifically includes in the context of this disclosure a salt formed by a physiologically tolerated acid, i.e. salts of a specific active compound with physiologically tolerated organic or inorganic acids-particularly when used on humans and/or mammals.

Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid.

The term “solvate” in accordance with this disclosure should be understood as meaning any form of a compound in accordance with the disclosure in which said compound is bonded by a non-covalent bond to another molecule (normally a polar solvent), including especially hydrates and alcoholates, like for example, methanolate. A preferred solvate is the hydrate.

Any compound referred to herein is intended to represent such specific compound as well as certain variations or forms. In particular, compounds referred to herein may have asymmetric centres and therefore exist in different enantiomeric or diastereomeric forms. Thus, any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same as, or different to, the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present disclosure Furthermore, any compound referred to herein may exist as tautomers. Specifically, the term tautomer refers to one of two or more structural isomers of a compound that exist in equilibrium and are readily converted from one isomeric form to another. Common tautomeric pairs are enamine-imine, amide-imidic acid, keto-enol, lactam-lactim, etc.

Unless otherwise stated, the compounds of the disclosure are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by 13C- or 14C-enriched carbon, or the replacement of at least one nitrogen by 15N-enriched nitrogen are within the scope of this disclosure.

The compounds of the disclosure or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrug.

As used herein, the terms “treat”, “treating” and “treatment” include the eradication, removal, reversion, alleviation, modification, or control of cholinergic system dysfunction after its onset. As used herein, the terms “prevention”, “preventing”, “preventive” “prevent” and “prophylaxis” refer to the capacity of a therapeutic to avoid, minimize, stall (the terms “prevention” and “prophylaxis” are used interchangeably herein) or difficult the onset or development of a disease or condition before its onset. Reference is made in particular to conditions related to cholinergic system dysfunction and more particularly to the reduction of density, or expression, and function of neuronal Nicotinic Acetylcholine Receptors. As such, the methods of the present disclosure also relate to situations where the condition is completely or partially inhibited, e.g., prevented from happening, or stopped, e.g., terminated, such that the subject no longer experiences the condition. Further, the present method includes both preventing and managing, or treating, the loss or the reduction in density, and or the reduction in function of neuronal Nicotinic Acetylcholine Receptors.

Cholinergic system dysfunction has been associated with the onset of neurodegenerative diseases. Altered expression levels of nAChRs or modified nAChRs expression and function in selected areas of the nervous system, have been described in connection to several neurodegenerative diseases such as Alzheimer's, Parkinson's and Huntington as well as in psychiatric disorders such as schizophrenia, and Amyotrophic Lateral Sclerosis-Parkinsonism/dementia Complex 1.

Reduced expression levels of neuronal Nicotinic Acetylcholine Receptors are associated with normal ageing and cognitive decline.

Therefore, by “treating” or “treatment” and/or “preventing” or “prevention”, as a whole, is meant at least a suppression or an amelioration of the symptoms associated with the condition afflicting the subject, where suppression and amelioration are used in a broad sense to refer to at least a 15 reduction in the magnitude of a parameter, e.g., symptom associated with the condition being treated, such as Mild Cognitive Impairment, Impaired Decision-Making disorders, executive functioning, Neuropsychiatric Symptoms, Mild Behavioral Impairment.

According to the present disclosure, a subject that is likely to suffer from a disease related to a dysfunction of the neuronal nicotinic acetylcholine receptor system, may be a normal (e.g., healthy) ageing individual, or otherwise an individual who is likely to suffer from a disease related to a dysfunction of the neuronal nicotinic acetylcholine receptor system based on a known family medical history.

In an embodiment of the disclosure, said potentiating effect induces the expression of chnrb2, neuronal nicotinic acetylcholine receptors, Cholinergic Receptor Nicotinic Beta 2 Subunit, or any other subunit of neuronal nicotinic acetylcholine receptors by a factor of 1.2, 1.5, 2, 3, 4 or more.

According to the present disclosure, diseases and conditions related to cholinergic system dysfunction, dysfunction of the neuronal nicotinic acetylcholine receptor system, expression of Nicotinic Acetylcholine Receptors, density of Nicotinic Acetylcholine Receptors, function of Nicotinic Acetylcholine Receptors reduction in the level of expression of Nicotinic Acetylcholine Receptors, and expression of the B2-nAChR include, but are not limited to, reduction in the density of neuronal nicotinic acetylcholine receptors in the brain, reduction in the density of cholinergic Receptor Nicotinic Beta 2 Subunit (B2-nAChR) in the brain, reduction in the density of chnrb2 in the brain, decision making impairment, age related cognitive decline, cognitive decline, age-related cognitive decline, memory loss, mild memory loss, age related memory loss, ageing, apathy, depression, mild behavioral impairment, Mild Cognitive Impairment, impairment of executive functions, cognitive decline, pain, neurodegenerative disorders, orthostatic hypotension, cholinergic system dysfunction, cholesterol homeostasis, neuropathic pain and neuropsychiatric symptoms.

By “neurological disorder” is meant any disease, condition and disorder of the central nervous system. Neurological disorders according to the present disclosure are, but not limited to, Alzheimer's disease, Parkinson's Disease, amyotrophic lateral sclerosis, epilepsy, autism, rett syndrome, Huntington's Disease, stroke, spinal cord injury, traumatic brain injury, Lewy body dementia, multiple sclerosis, Pick's disease, Niewmann-Pick disease, Creutzfeld-Jakob disease, Guillain-Barre syndrome, Bell's palsy, diabetic neuropathy, amyotrophic lateral sclerosis, amyloid angiopathy, cerebral amyloid angiopathy, systemic amyloidosis, Petit Mal Epilepsy, Temporal Lobe Epilepsy, Post-Traumatic Epilepsy, Generalized Seizure Disorder, all forms of Epilepsy, Postherpetic Neuralgia, Viral Meningitis, Viral Encephalitis, Acute Poliomyelitis, Postpolio Syndrome, HIV Dementia, HIV-associated Myelopathy, HIV-associated Peripheral Neuropathy, HTLV-1 Myelopathy, Influenza, Trigeminal Neuralgia, Reyets Syndrome, Meniere's Disease, Reflex Sympathetic Dystrophy, Cerebrovascular Disease, Prophylaxis of Brain Injury caused by Stroke, Chronic Inflammatory Demyelinating Polyneuropathy, major depression, anxiety-related disorders, post-traumatic stress disorder, supranuclear palsy, feeding disorders, fibromyalgia, single episode depression, recurrent depression, child abuse induced depression, and postpartum depression; dysthemia; bipolar disorders; cyclothymia; fatigue syndrome; stress-induced headache, hereditary cerebral hemorrhage with amyloidosis of the Dutch type, inclusion body myositis, mild cognitive impairment, complications due to stroke, head trauma, or spinal injury, or other injuries to the brain, peripheral nervous, central nervous, or neuromuscular system, acute spinal cord and brain injury, demyelinating diseases such as multiple sclerosis, pain, acute pain, chronic pain, neuropathic pain, acute neuropathic pain, chronic neuropathic pain, migraine, post herpetic neuralgia, neuritis, temporomandibular disorder, myofascial pain, back pain, diabetic neuropathy, numbness, tingling sensation, trauma or injury to a nerve, allodynia, dystonia, Leber's hereditary optic neuropathy (LHON), schizophrenia.

By “Neuropathic pain” is meant pain associated with damage or permanent alteration of the peripheral or central nervous system. Clinical manifestations of neuropathic pain include a sensation of burning or electric shock, feelings of bodily distortion, allodynia and hyperpathia.

In some embodiments of the present disclosure, expression, or density, of cholinergic Receptor Nicotinic Beta 2 Subunit (B2-nAChR), expression, or density, of chnrb2, or expression, or density, of Nicotinic Acetylcholine Receptor, refers to said expression, or said density, of said nAChR, and said B2-nAChR in the CNS. More specifically, the expression, or density, of cholinergic Receptor Nicotinic Beta 2 Subunit (B2-nAChR), or expression, or density, of Nicotinic Acetylcholine Receptor, refers to said expression, or said density, of said nAChR, and said B2-nAChR in the brain. Even more specifically, expression, or density, of cholinergic Receptor Nicotinic Beta 2 Subunit (B2-nAChR), or expression, or density, of Nicotinic Acetylcholine Receptor, refers to said expression, or said density, of said nAChR, and said B2-nAChR in the brain cortical and subcortical regions.

The terms “expression”, “expression level/s”, or “density” are used interchangeably herein and generally refer to the number of copies of the protein of interest (e.g., B2-nAChR, nAChR, or chnrb2) in the tissues/cells of interest (e.g., CNS, brain, or brain cortical and subcortical regions).

In some embodiments, By enhancing the expression, or density as referred to the cholinergic Receptor Nicotinic Beta 2 Subunits (B2-nAChR), chnrb2, or the nicotinic acetylcholine receptors, according to the present disclosure, is generally meant generating an increase in the number of cholinergic Receptor Nicotinic Beta 2 Subunits (B2-nAChR), chnrb2, or the nicotinic acetylcholine receptors wherein the by said increase is meant an amount greater by at least 5%, I 0%, 25%, 50%, I 00%, 150%, 200%, 500%, 1000%, or more.

The Cholinergic system dysfunction is a dysfunction of the Nicotinic Acetylcholine receptors system characterized by reduction in the density expression and function of the neuronal Nicotinic Acetylcholine receptors, in selected areas of the nervous system.

Any of the compositions of the present disclosure may also comprise a pharmaceutically accepted salt and/or at least one excipient. In some embodiments, the compositions further comprise one or more pharmaceutically acceptable salts or excipients. Pharmaceutically accepted salts or excipients are further discussed below. The terms “composition” and “pharmaceutical composition” are used interchangeably herein.

A composition according to the present disclosure is generally a composition which comprises at least one compound selected from the compounds of general formula (I), as described and illustrated herein, and described and illustrated in FIG. 1. In some embodiments, the said selected compound according to general formula (I) is deuterated analog of 2-[(1R)-1-phenyl-3-[(1-phenylethyl)amino]propyl]phenol; 2-[(1S)-1-phenyl-3-[(1-phenylethyl)amino]propyl]phenol; or 3,3-diphenyl-N-(1-phenylethyl)propan-1-amine. In some embodiments, the said selected compound according to general formula (I) is 3,3-diphenyl-N-(1-phenylethyl)propan-1-amine.

Any of the compounds of Table(1), FIG. 1, derivatives of the compounds of Table(1), or the compounds described in the present specification may be employed in the compositions and methods of the present disclosure. The term “compound according to formula (I)” refers to any of the compounds depicted, or described, in Table(1), FIG. 1, and the present specification. The terms “formula 1” and “general formula 1” are used interchangeably herein.

Any of the compounds employed in the methods and compositions of the present disclosure may contain one or more asymmetrically-substituted carbon or nitrogen atoms, and may be isolated in optically active or racemic form. Thus, all chiral, diastereomeric, racemic form, epimeric form, and ail geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The compounds can be formulated as a mixture of one or more diastereomers. Alternatively, the diastereomers can be separated and one or more of the diastereomers can be formulated individually. The chiral centers of the compounds of the present disclosure can have the S or the R configuration, as defined by the IUPAC 1974 Recommendations. For example, mixtures of stereoisomers may be separated using the techniques taught in the Examples section below, as well as modifications thereof.

Atoms making up the compounds of the present disclosure are intended to include all isotopic forms of such atoms. Compounds employed in the present disclosure include those with one or more atoms that have been isotopically modified or enriched, in particular those with pharmaceutically acceptable isotopes or those useful for pharmaceutically research. Isotopes, as used herein, include those atoms having the same atomic number but different mass numbers. By way of general example and without limitation, isotopes of hydrogen include deuterium and tritium, and isotopes of carbon include 13C and 14C. Similarly, it is contemplated that one or more carbon atom(s) of a compound of the present disclosure may be replaced by a silicon atom(s). Furthermore, it is contemplated that one or more oxygen atom(s) of a compound of the present disclosure may be replaced by a sulfur or selenium atom(s).

Compounds employed in the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may. if desired, be delivered in prodrug form. Thus, the disclosure contemplates prodrugs of compounds of the present disclosure as well as methods of delivering prodrugs.

Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this disclosure is not critical, so long as the salt, as a whole, is pharmacologically acceptable.

Additional examples of pharmaceutically acceptable salts and their methods of preparation and use are presented in Handbook of Pharmaceutical Salts: Properties, and Use (2002), which is incorporated herein by reference.

A subject in need according to the present disclosure is a subject having, without limitation, any of the following disorders, diseases or conditions: a neurological disorder, dementia, a nervous system-related tumor, cancer, diabetes, brain cancers, glioblastomas, retinoblastomas, pediatric neuroblastoma, glioblastoma, Brain Trauma Injuries, Metabolic diseases, obesity, Renal Fibrosis, Myelination disorder, Multiple Sclerosis, chronic wounds, non-healing wounds, skin disorders, or is in need of increased stem cell regeneration.

The compositions of the disclosure may include one or more of the compounds of the present disclosure, which may be present in amount(s) of at least 1, 5, 10, 25, 50, 100, 250, 500, or 750 μg; at least 1, 2, 5, 10, 25, 50, 100, 250, 500, or 750 mg; or at least 1, 2, 5, 10, 15, 20, 25, 50, or 100 g, or any range between these values. The compositions may include at least 0.01%, 0.05%, 0.1%, 0.5%, 1%, 2%, 3%, 5%, 8%, 10%, 15%, 20%, 25%, 40%, 50%, 60%, 75%, 85%, 90%, 95%, 98%, or 99% of the compounds of the present disclosure, or any range between these values. In certain embodiments, the compositions include less than 99%, 98%, 95%, 90%, 85%, 75%, 60%, 50%, 40%, 25%, 20%, 15%, 8%, 5%, 3%, 2%, 1%, or 0.5% of the compounds of the present disclosure. In compositions containing two or more compounds of the present disclosure, the ratio between any two of the said compounds presents may be at least 1000:1, 500:1, 100:1, 50:1, 25:1, 10:1, 5:1, 3:1, 2:1, 1:1, 1:2, 1:3, 1:5, 1:10, 1:25, 1:50, 1:100, 1:500, or 1:1000, or any range between these values.

The compound or compositions of the disclosure are given on a per diem basis but should not be interpreted as necessarily being administered on a once daily frequency. Indeed, the compositions, compound, salt or prodrug thereof, can be administered at any suitable frequency, for example as determined conventionally by a physician taking into account a number of factors, but typically about four times a day, three times a day, twice a day, once a day, every second day, twice a week, once a week, twice a month or once a month. In some situations a single dose may be administered, but more typically administration is according to a regimen involving repeated dosage over a treatment period. In such a regimen the daily dose and/or frequency of administration can, if desired, be varied over the course of the treatment period, for example introducing the subject to the compound, composition, salt or prodrug thereof at a relatively low dose and then increasing the dose in one or more steps until a full dose is reached.

The treatment period is generally as long as is needed to achieve a desired outcome.

It will generally be found preferable to administer the (Active Pharmaceutical Ingredient-compounds and compositions of the present disclosure) API in a pharmaceutical composition that comprises the API and at least one pharmaceutically acceptable excipient. The excipient(s) collectively provide a vehicle or carrier for the API. Pharmaceutical compositions adapted for all possible routes of administration are well known in the art and can be prepared according to principles and procedures set forth in standard texts and handbooks such as those individually cited below
USIP, ed. (2005) Remington: The Science and Practice of Pharmacy, 21st ed., Lippincott, Williams & Wilkins.
Allen et al. (2004) Ansel's Pharmaceutical Dosage Forms and Drug Delivery Systems, 8th ed., Lippincott, Williams & Wilkins.
Suitable excipients are described, for example, in Kibbe, ed. (2000) Handbook of Pharmaceutical Excipients, 3rd ed., American Pharmaceutical Association.

Examples of formulations that can be used as vehicles for delivery of the API in practice of the present disclosure include, without limitation, solutions, suspensions, powders, granules, tablets, capsules, pills, lozenges, chews, creams, ointments, gels, liposomal preparations, nanoparticulate preparations, injectable preparations, enemas, suppositories, inhalable powders, sprayable liquids, aerosols, patches, depots and implants.

For oral delivery, the API can be formulated in liquid or solid form, for example as a solid unit dosage form such as a tablet or capsule. Such a dosage form typically comprises as excipients one or more pharmaceutically acceptable diluents, binding agents, disintegrants, wetting agents and/or antifrictional agents (lubricants, anti-adherents and/or glidants). Many excipients have two or more functions in a pharmaceutical composition. Characterization herein of a particular excipient as having a certain function, e.g., diluent, binding agent, disintegrant, etc., should not be read as limiting to that function.

The compositions of the disclosure may be in any of the forms described herein. In some embodiments, the composition is food that has been enriched with one or more of the compounds of the present disclosure.

By “treating” is meant ameliorating at least one symptom of a condition or disease in a subject having the condition or disease (e.g., a subject diagnosed with a neurological disorder), as compared with an equivalent untreated control. Such reduction in the symptom (e.g., a reduction in blood glucose levels) is at least 5%, 10%, 20%, 40%, 50%, 60%, 80%, 90%, 95%, or 100%, as measured by any standard technique.

By “treating prophylactically” is meant to reduce the frequency of disease occurrence or severity of disease upon its onset by administering to the subject a therapeutic prior to onset of the disease. Prophylactic treatment can include disease prevention. Subjects at higher risk of developing metabolic disorders or IBD (e.g., risk factors described herein) may be treated prophylactically in the methods of the disclosure.

An “agonist” is defined as a compound that binds to a receptor and has an intrinsic effect, and thus, increases the basal activity of a receptor when it contacts the receptor.

An “antagonist” is defined as a compound that competes with an agonist or inverse agonist for binding to a receptor, thereby blocking the action of an agonist or inverse agonist on the receptor.

However, an antagonist (also known as a “neutral” antagonist) has no effect on constitutive receptor activity. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding.

A “partial antagonist” is defined as a compound that binds to the receptor and generates an antagonist response; however, a partial antagonist does not generate the full antagonist response.

Partial antagonists are weak antagonists, thereby blocking partially the action of an agonist or inverse agonist on the receptor.

An “inverse agonist” is defined as a compound that produces an effect opposite to that of the agonist by occupying the same receptor and, thus, decreases the basal activity of a receptor (i.e., signalling mediated by the receptor). Such compounds are also known as negative antagonists.

An inverse agonist is a ligand for a receptor that causes the receptor to adopt an inactive state relative to a basal state occurring in the absence of any ligand. Thus, while an antagonist can inhibit the activity of an agonist, an inverse agonist is a ligand that can alter the conformation of the receptor in the absence of an agonist.

The term “excipient” refers to components of a drug compound other than the active ingredient (definition obtained from the European Medicines Agency—EMA). They preferably include a “carrier, adjuvant and/or vehicle”. Carriers are forms to which substances are incorporated to improve the delivery and the effectiveness of drugs. Drug carriers are used in drug-delivery systems such as the controlled-release technology to prolong in vivo drug actions, decrease drug metabolism, and reduce drug toxicity. Carriers are also used in designs to increase the effectiveness of drug delivery to the target sites of pharmacological actions (U.S. National Library of Medicine. National Institutes of Health). Adjuvant is a substance added to a drug product formulation that affects the action of the active ingredient in a predictable way. Vehicle is an excipient or a substance, preferably without therapeutic action, used as a medium to give bulk for the administration of medicines (Stedman's Medical Spellchecker, 2006 Lippincott Williams & Wilkins). Such pharmaceutical carriers, adjuvants or vehicles can be sterile liquids, such as water and oils, including those of petroleum, animal, vegetable or synthetic origin, such as peanut oil, soybean oil, mineral oil, sesame oil and the like, excipients, disgregants, wetting agents or diluents. Suitable pharmaceutical carriers are described in “Remington's Pharmaceutical Sciences” by E. W. Martin. The selection of these excipients and the amounts to be used will depend on the form of application of the pharmaceutical composition.

The pharmaceutical composition used according to the present disclosure can be adapted to any form of administration, be it orally or parenterally, for example pulmonarily, nasally, rectally and/or intravenously. Therefore, the formulation according to the present disclosure may be adapted for topical or systemic application, particularly for dermal, subcutaneous, intramuscular, intra-articular, intraperitoneal, pulmonary, buccal, sublingual, nasal, percutaneous, vaginal, oral or parenteral application. The preferred form of rectal application is by means of suppositories.

Suitable preparations for oral applications are tablets, pills, chewing gums, capsules, granules, drops or syrups. Suitable preparations for parenteral applications are solutions, suspensions, reconstitutable dry preparations or sprays.

The composition of the disclosure may be formulated as deposits in dissolved form or in patches, for percutaneous application. Skin applications include ointments, gels, creams, lotions, suspensions or emulsions.

The composition of the disclosure may be formulated for its simultaneous, separate or sequential administration, with at least a pharmaceutically acceptable excipient. This has the implication that the composition of the composition comprising of a compound of general formula (I) may be administered:

As a composition that is being part of the same medicament formulation, both being then administered always simultaneously.

As a composition of two units, each with one of them giving rise to the possibility of simultaneous, sequential or separate administration.

By an “effective” amount or a “therapeutically effective amount” of a drug or pharmacologically active agent is meant a nontoxic but sufficient amount of the drug or agent to provide the desired effect. In the present disclosure, an “effective amount” of a component of the composition is the amount of that compound that is effective to provide the desired therapeutic effect. The amount that is “effective” will vary from subject to subject, depending on the age and general condition of the individual, the particular active agent or agents, and the like. Thus, it is not always possible to specify an exact “effective amount”. However, an appropriate “effective” amount in any individual case may be determined by one of ordinary skill in the art using routine experimentation.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth.

The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

### REFERENCES

1. c. a. barnes, j. meltzer, f houston, g. orr, k. mcgann and g. l. wenk Chronic treatment of old rats with donepezil or galantamine: effects on memory, hippocampal plasticity and nicotinic receptors, neuroscience vol. 99, no. 1, pp. 17-23, 2000.
2. Gahring L C, Vasquez-Opazo G A, Rogers S W. Choline Promotes Nicotinic Receptor a4+B2 Up-regulation. The Journal of Biological Chemistry. 2010; 285(26):19793-19801. doi:10.1074/jbc.M110.108803.
3. James Wilkinson a, Dennis Foretia b, Steven Rossington a, Anthony Heagerty b, 2′-Hydroxy-fendiline analogues as potent relaxers of isolated arteries, European Journal of Pharmacology 561 (2007) 160-163.
4. Jun Go, Thi-Kim-Quy Ha, Ji Yeon Seo, Tae-Shin Park, Young-Kyoung Ryu, Hye-Yeon Park, Jung-Ran Noh, Yong-Hoon Kim, Jung Hwan Hwang, Dong-Hee Choi, Dae Youn Hwang, Sanghee Kim, Chul-Ho Lee, Won Keun Oh, Kyoung-Shim Kim, Piperlongumine activates Sirtuin1 and improves cognitive function in a murine model of Alzheimer's disease, Journal of Functional Foods, Volume 43, 2018, Pages 103-111, ISSN 1756-4646.
5. Kerchner et al, 2010, Bapineuzumab, Expert Opin Biol Ther., 10(7):1121-1130.
6. Mitsis E M, Cosgrove K P, Staley J K, et al. Age-related decline in nicotinic receptor availability with [1231]5-IA-85380 SPECT. Neurobiology of aging. 2009; 30(9): 1490-1497. doi:10.1016/j.neurobiolaging.2007.12.008.
7. TargetScan Release 7.1
8. Molecular identity of the o2 receptor, Assaf Alon, Hayden R. Schmidt, Michael D. Wood, James J. Sahn, Stephen F. Martin, Andrew C. Kruse, Proceedings of the National Academy of Sciences July 2017, 114 (27) 7160-7165; DOI:10.1073/pnas.1705154114.
9. Progress in Medicinal Chemistry, Volume 57. Rouillard A D, Gundersen G W, Fernandez N F, Wang Z, Monteiro C D, McDermott M G, Ma'ayan A. The harmonizome: a collection of processed datasets gathered to serve and mine knowledge about genes and proteins. Database (Oxford). 2016 Jul. 3; 2016. pii: baw100.
10. Sahn J J, Mejia G L, Ray P R, Martin S F, Price T J. Sigma 2 Receptor/Tmem97 Agonists Produce Long Lasting Antineuropathic Pain Effects in Mice. ACS chemical neuroscience. 2017; 8(8): 1801-1811. doi:10.1021/acschemneuro.7b00200.
11. Yi B, Sahn J J, Ardestani P M, et al. Small molecule modulator of sigma 2 receptor is neuroprotective and reduces cognitive deficits and neuro-inflammation in experimental models of Alzheimer's disease. Journal of neurochemistry. 2017; 140(4):561-575. doi:10.1111/jnc.13917.

1. A pharmaceutical composition comprising a therapeutically effective amount of at least one compound of general formula (I).

2. The pharmaceutical composition of claim 1, wherein the compound of general formula (I) is selected from:
deuterated analog of 2-[(1R)-1-phenyl-3-[(1-phenylethyl)amino]propyl]phenol;
2-[(1S)-1-phenyl-3-[(1-phenylethyl)amino]propyl]phenol; and
3,3-diphenyl-N-(1-phenylethyl)propan-1-amine.

3. The pharmaceutical composition of claim 1, wherein the selected compound is 3,3-diphenyl-N-(1-phenylethyl)propan-1-amine.

4. A method of treatment of a subject in need, wherein said subject is suffering from a disease related to a dysfunction of the neuronal nicotinic acetylcholine receptor system, wherein the method comprises administering to said subject a therapeutically effective amount of the pharmaceutical composition of claim 1.

5. A method of providing prophylaxis to a subject, wherein said subject is likely to suffer from a disease related to a dysfunction of the neuronal nicotinic acetylcholine receptor system, and wherein the method comprises administering to said subject a therapeutically effective amount of the pharmaceutical composition of claim 1.

6. The method of claim 4, wherein said disease related to a dysfunction of the neuronal nicotinic acetylcholine receptor system is a reduction in the density of neuronal nicotinic acetylcholine receptors in the brain.

7. The method of claim 5, wherein said disease related to a dysfunction of the neuronal nicotinic acetylcholine receptor system is a reduction in the density of neuronal nicotinic acetylcholine receptors in the brain.

8. The method of claim 4, wherein said disease related to a dysfunction of the neuronal nicotinic acetylcholine receptor system is age related cognitive decline.

9. The method of claim 5, wherein said disease related to a dysfunction of the neuronal nicotinic acetylcholine receptor system is age related cognitive decline.

10. The method of claim 6, wherein said disease related to a dysfunction of the neuronal nicotinic acetylcholine receptor system is age related cognitive decline.

11. The method of claim 7, wherein said disease related to a dysfunction of the neuronal nicotinic acetylcholine receptor system is age related cognitive decline.

12. The method of claim 4, wherein the said subject is in need of cognitive enhancement during reinforced learning, working tasks, and decision-making processes.

13. The method of claim 5, wherein the said subject is in need of cognitive enhancement during reinforced learning, working tasks, and decision-making processes.

14. The method of claim 6, wherein the said subject is in need of cognitive enhancement during reinforced learning, working tasks, and decision-making processes.

15. The method of claim 7, wherein the said subject is in need of cognitive enhancement during reinforced learning, working tasks, and decision-making processes.
